(19) United States
(12) Patent Application Publication
(10) Pub. No.: US 2025/0090716 A1
(43) Pub. Date: Mar. 20, 2025

(54) Thimble Bank

(71) Applicant: Yaron Abraham, Los Angeles, CA (US)

(72) Inventor: Yaron Abraham, Los Angeles, CA (US)

(21) Appl. No.: 18/467,596

(22) Filed: Sep. 14, 2023

(51) Int. Cl. A61L 15/44; A61F 13/00; A61L 15/18; A61L 15/22; A61L 15/42; A61L 15/52; A61L 15/58

(52) U.S. Cl. CPC A61L 15/44; A61F 13/00063; A61F 13/00085; A61L 15/18; A61L 15/225; A61L 15/425; A61L 15/52; A61L 15/58; A61F 2013/00285; A61F 2013/0048; A61F 2013/00578; A61F 2013/00604; A61F 2013/00655; A61F 2013/00778; A61F 2013/00889

(57) ABSTRACT

A wrap-around bandage for body tips, including fingers, toes, and other similar-shaped body parts wherein at least one section of the bandage wraps around the body-part to secure the bandage.

### CROSS REFERENCE TO RELATED APPLICATIONS

This application claims the benefit of U.S. Provisional Patent Application No. 63/406,637, filed Sep. 14, 2022, entitled “Wrap Bandage”, the disclosure of which is incorporated by reference herein in its entirety.

### FIELD OF THE INVENTION

The present invention relates to a wrap-around bandage for body tip parts, including digits.

### BACKGROUND

Most bandages are flat and elongated with an absorbent middle section to collect leaking bodily fluids or hold bactericide or other medicaments. Most typically, the two elongate portions, each opposite from each other, being on either side of the distinct absorbent middle section, have a type of adhesive to fasten the bandage and hold the absorbent middle section to the skin, thereby covering and protecting the wound.

As is well known, but the many differently shaped bandages developed over time, this flat bandage can be attached to most sections of the body to cover and protect a wound, but this flat bandage seldom fits any part of the body well, especially when the body is shaped in a curvilinear fashion. The flat bandage binds, puckers, bunches, and otherwise only somewhat accomplishes the goal of keeping the wound protected. Attempts have been made to make the bandage from elastomeric materials, which improve the fit of curvilinear body portions but continue to suffer from the same shortcomings due to the shape of the body and the bandage.

Some flat bandages have incorporated multiple elongate shapes, such as those in the shape of the letter “H” in order to have multiple portions to wrap around tricky spots of the body, such as a fingertip. In this design, the legs of the “H” are used to wrap the body tip, such as a digit like a toe or finger. This design is an improvement over the traditional bandage (in the shape of an ellipse with a parallel middle portion), but, when used on a fingertip, toe tip, nose tip, or other similarly shaped body parts (herein, a “body tip”), continues to suffer from the aforementioned shortcomings.

Some efforts to conform the bandage to the body tip in the art contain a parabolic shape, which can slip on a body tip just as well as slip right off the body tip, especially if liquids, particulate solids, or other issues (such as heat) reduce the ability of the adhesive to keep the bandage in place. Still other styles of bandages for body tips have incorporated strands to tie in place (not unlike a shoelace) or a fingertip bandage with multiple straps for the final segment (above the last knuckle) of a finger and fingertip. The single goal of all of these fingertip bandages is to cover the wound or other issue for the area to protect the wounded area, regardless of whether the bandage has adhesive strands, tying strands, various metal and plastic fastening devices to hold the bandage in place once it is wrapped around the affected area, or no strands and only adhesive. As an alternative to a bandage, sometimes skin glue, staples or stitches are used to protect a wound. A combination of skin glue, stitches, staples or bandages can be used as well.

### SUMMARY OF THE INVENTION

It is therefore an object of the present invention to provide a bandage for protecting wounds.

It is another object of the invention to provide a bandage for protecting wounds on a body tip of the user, such as a digit like a finger or toe.

It is yet another object of the invention to provide a fastening means to secure the bandage to a body tip of the user.

It is another object of the invention to pull together the edges of a skin wound to facilitate healing and reduce chances of infection.

### BRIEF DESCRIPTION OF THE DRAWINGS

FIG. 1 is a frontal side view to illustrate the bandage with the flap adhered to the Sleeve second end portion (sometimes referred to as “closed”).

FIG. 2 is a backside view to illustrate where the closed flap proceeds from the Sleeve second end portion.

FIG. 3 is an oblique bottom view to illustrate a closed flap and the inner face surface.

FIG. 4 is a frontal side view to illustrate the bandage with the flap not adhered to the Sleeve second end portion (sometimes referred to as “open”).

FIG. 5 is a frontal side view to illustrate the flap partially closed and the protective shield over the adhesive of the flap.

FIG. 6 is a backside view to illustrate where the open flap proceeds from the Sleeve second end portion.

FIG. 7 is a diagonal and oblique bottom view to illustrate an open flap with a fastening means, such as an adhesive in the inner surface of the flap, and the inner face surface of the Sleeve.

FIG. 8 is an oblique bottom view to illustrate the outer surface of an open flap and the inner face surface of the Sleeve.

FIG. 9 is an oblique bottom view to illustrate an open flap with a fastening means, such as a hook and loop closure in the inner surface of the flap and outer surface of the Sleeve second end portion.

FIG. 10 is a frontal side view to illustrate an open flap with a fastening means, such as an adhesive on the inner surface of the flap and outer surface of the Sleeve second end portion, and the inner face surface of the Sleeve.

FIG. 11 is a frontal side view to illustrate an open flap and an absorbent pad on part of the inner face surface of the Sleeve.

FIG. 12 is a frontal side view to illustrate an open flap and a medicament on part of the inner face surface of the Sleeve.

FIG. 13 is a frontal side view to illustrate the flap as a compression cuff in an inside out orientation before pulling the flap down over the Sleeve second end portion.

FIG. 14 is a frontal side view to illustrate the flap as a compression cuff in an inside out and rolled up orientation before pulling the flap down over the Sleeve second end portion.

FIG. 15 is a frontal side view to illustrate the flap as a compression cuff in an right side out orientation after pulling the flap down over the Sleeve second end portion.

FIG. 16 is a frontal side view to illustrate the flap as a compression cuff in an right side out orientation after pulling the flap down over the Sleeve second end portion and then said flap extending just beyond the Sleeve second end portion and onto part of the skin of the user.

### DETAILED DESCRIPTION

The present invention presents a bandage for protecting wounds and assisting in the healing of wounds on a toe, finger, nose tip, or other protruding body parts (a “body tip”).

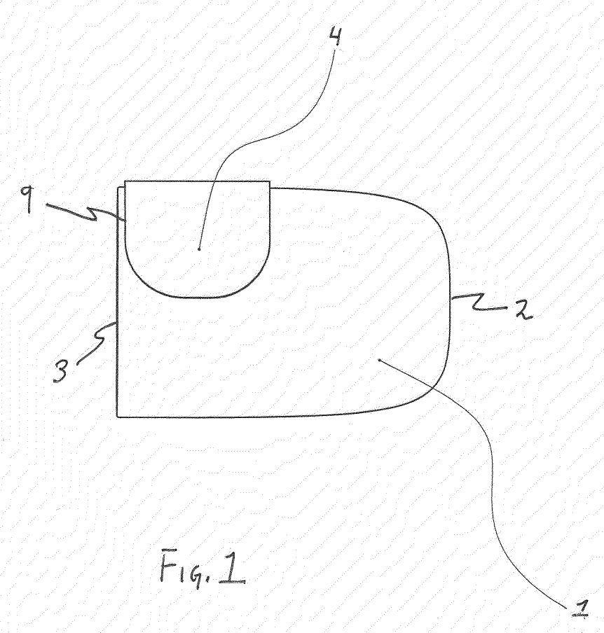

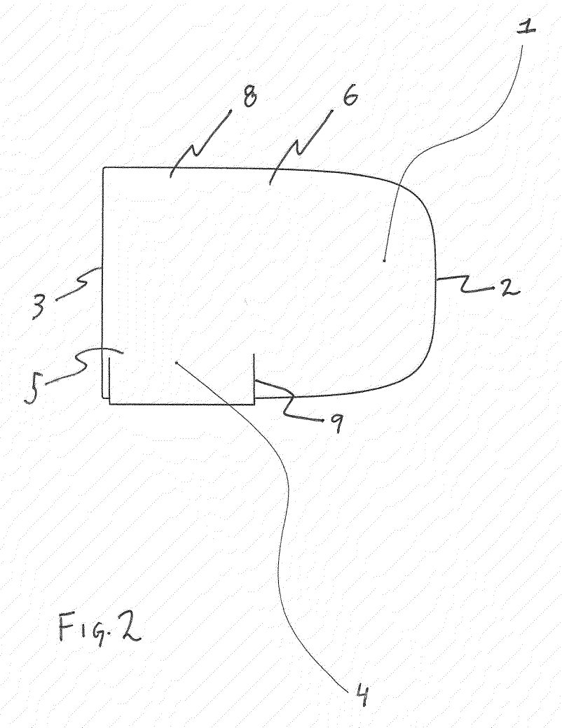

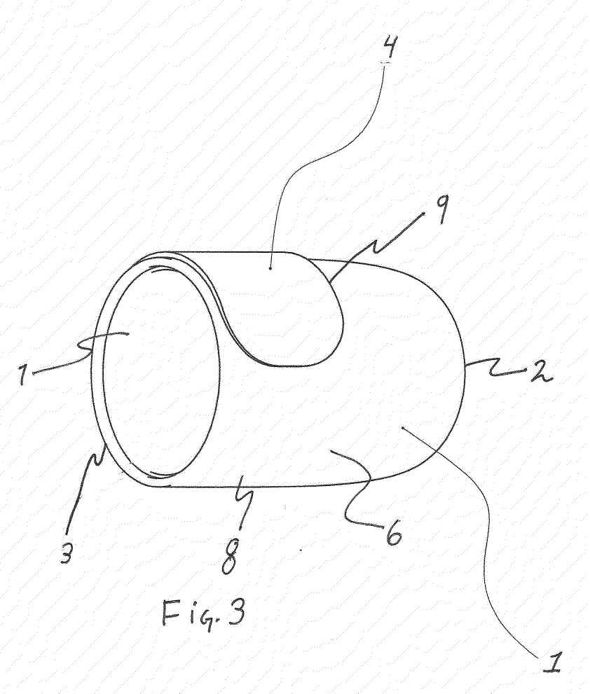

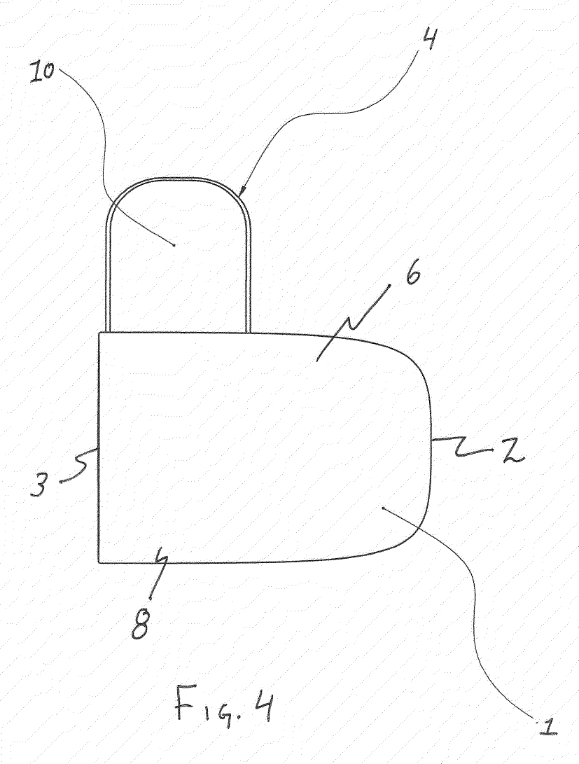

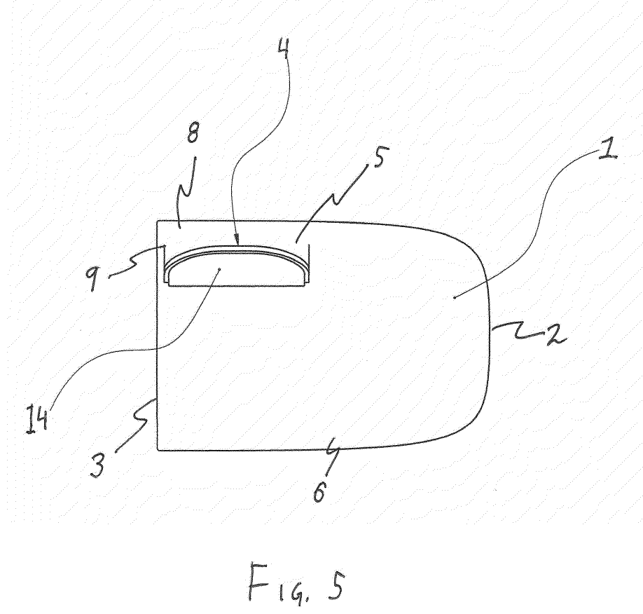

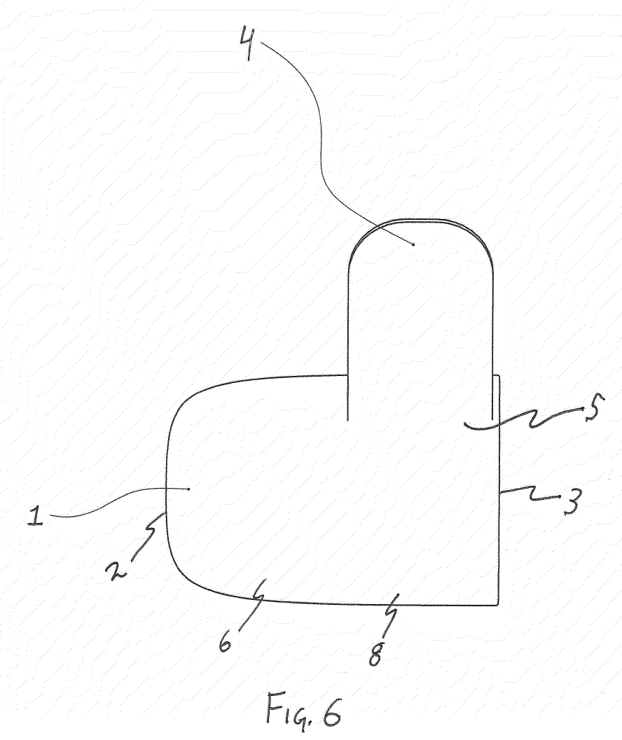

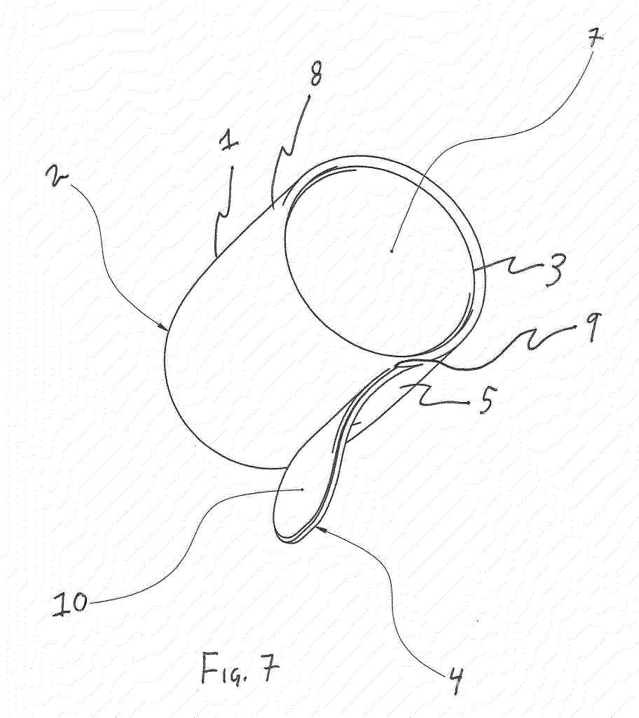

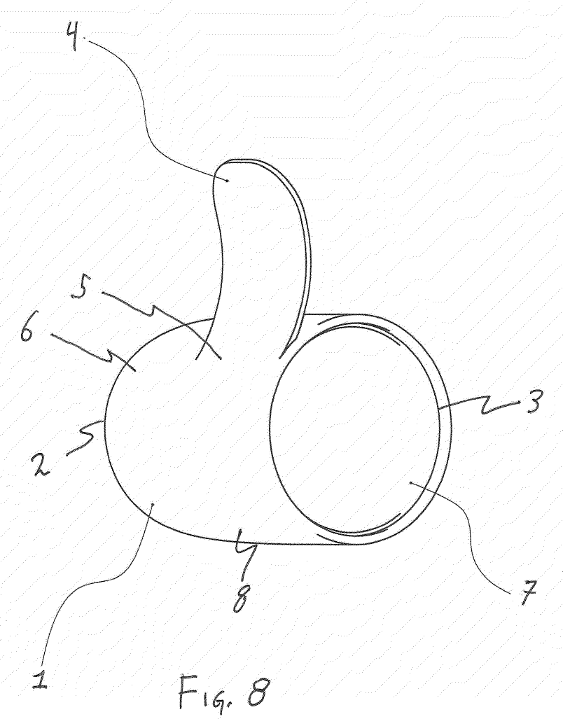

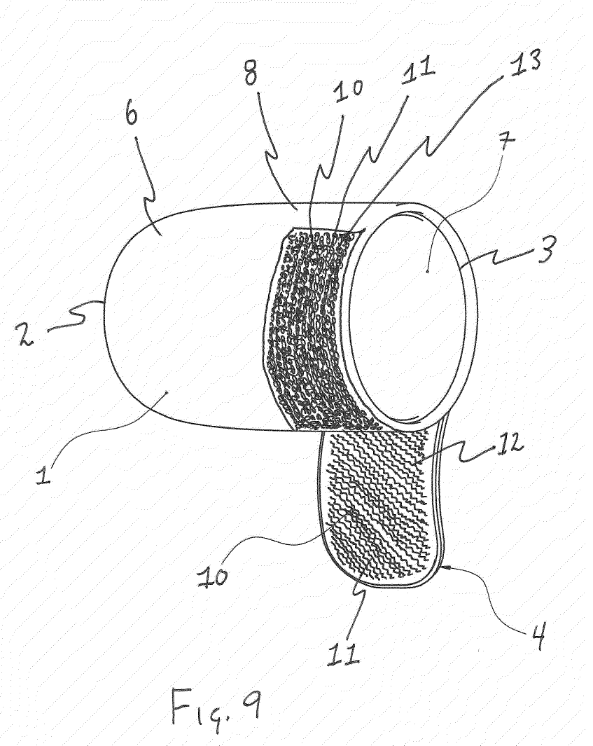

Now referring to FIGS. 1 though 8, the present protective bandage invention is comprised of a “Sleeve” (1) with one closed end (2), an opposing open end (3), and made of a pliable material. The Sleeve has an outer surface (6) and an inner face surface. (7) The exterior portion (“outer surface” (6)) of the Sleeve (1) also contains a hingeabily-attached flap. (4) In the preferred embodiment, the flap (4) is generally in a rectangular shape.

The Sleeve (1) with a closed-end (2) may take the general form of several shapes, such as a conical frustum with a closed narrow end, a blunted tangent ogive, an elliptical cone, a blunted parabolic cone, a cylinder with a closed-end, and any other curvilinear shape designed to conform to a body tip.

The preferred embodiment of the invention is a parabolic-shaped Sleeve (2) that is designed to fit over a body tip. The length of the Sleeve, therefore, is preferably sufficient to at least partially cover the body tip, but the length of the protective bandage Sleeve (1) may vary in different embodiments. Additionally, the Sleeve (1) may be sized differently depending upon which body tip, including digits, is meant to be fitted and covered. Therefore, the widths and lengths of the Sleeve (1) may vary depending upon the body tip that is to be covered. The Sleeve (1) is preferably made of an elastomeric material to reduce binding and puckering, but various embodiments may be made of different material or even a plurality of materials. The Sleeve (1) is preferably formed from a material selected from the group consisting of: plastic, polyvinyl chloride, polyethylene, polyurethane, latex, woven fabric, rayon, nylon, or a combination thereof.

In the preferred embodiment, one side of the flap (4) is tangentially attached (5) to one side of the outer surface portion of the Sleeve (1) in the second end portion. (8) The flap (4) attachment is generally in a curved planar to the side portions of the parabolic-shaped Sleeve (1), in some embodiments tangentially attached to the Sleeve (1), and generally perpendicularly orientated to the tip of the parabolic-shaped Sleeve (1) (and thereby parallel to the directrix). Preferably, the flap (4) is of the same material and thickness as the Sleeve. (1) In this embodiment, the remaining three sides of the flap (4) are not attached to the Sleeve (1); only the single attached side is attached (5) to the Sleeve. (1) The flap (4), attached (5) by a single side, serves to function as a strap hinge to fold over and onto the outer surface (6) (sometimes referred to herein as the “outside portion”) of the Sleeve. (1) The portion of the Sleeve (1) and the flap (4) that become in contact with each other when the flap (4) is folded over onto the Sleeve (1) will be referred as the “Connection” (9) for convenience. The Connection (9), therefore, consists of a Sleeve (1) portion and a flap (4) portion. The Sleeve (1) portion and the flap (4) portion of the Connection (9), in the preferred embodiment, incorporate a fastening method. (10)

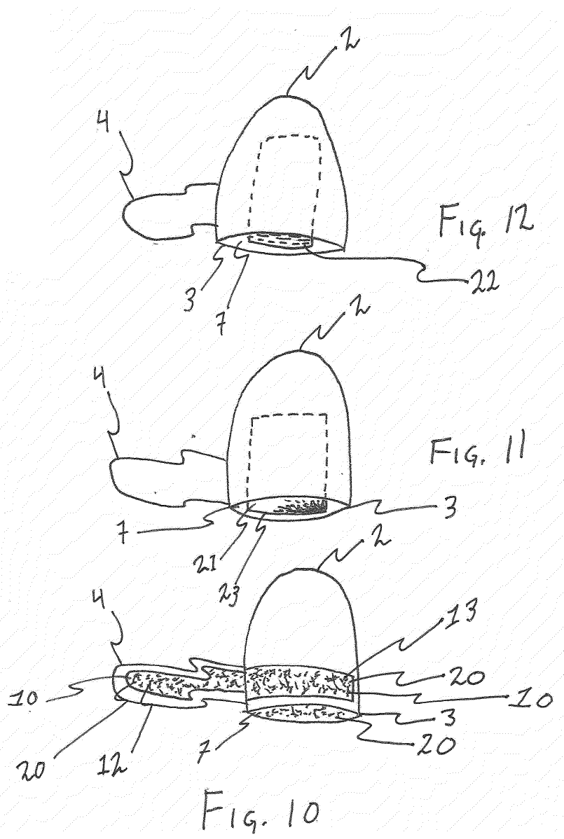

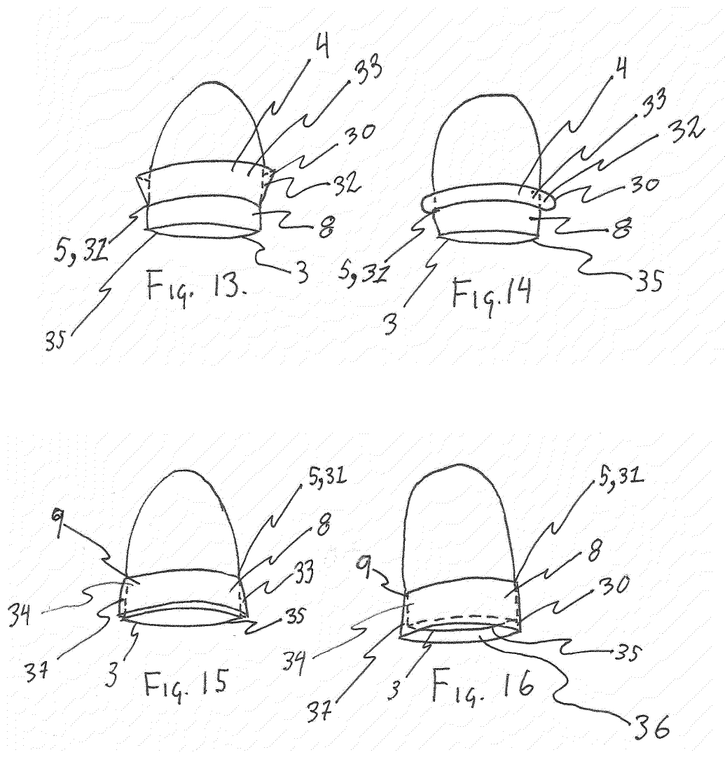

Now referring to FIGS. 13 trough 16, and in other embodiments, the flap (4) has other shapes. In one such embodiment, the flap (4) circumscribes (30) the second end portion (8) of the Sleeve (1) and is attached (5) (31) along the distal edge of the flap (4) in said second end portion (8) of the Sleeve. (1) Before application to a body tip using the flap (4) to help secure the Sleeve (1) bandage in place, the flap (4) of this embodiment is retained in an inside out orientation (32) with its interior portion (33) facing outward and its outside portion (34) facing inwards, flipped upward away from the opening. (3) This may involve folding the flap (4) in this orientation (see FIG. 13) or rolling the flap (4) in this open orientation (see FIG. 14). This attachment method enables the user to pull (see FIG. 15) or roll down (see FIG. 16) the flap (4), dependent upon the open flap (4) orientation, over the second end portion (8) of the Sleeve (1) towards the opening (3) of the Sleeve. (1) This action results in the flap (4) in the closed position and thereby helping to secure the Sleeve (1) bandage to the body tip. In another embodiment, the flap (4) extends beyond (36) the terminal end (35) of the Sleeve (1) second end portion (8) and contacts the skin of the user. The result is that the flap (4) in this embodiment, regardless of the open orientation or closed position relative to the Sleeve (1) second end portion (8), serves as a skirt (rather than a strap) around the outer surface (6) of the Sleeve (1) second end portion (8) to a right-side-out (37) and closed orientation.

In sum, the flap (4), when pulled over and connected to the outer surface (6) of the Sleeve (1) at the Connection (9) is designed to further secure the preferred embodiment over the body tip. In the preferred embodiment, the Connection (9) comprises approximately of one-half of the side portion of the parabolic-shaped Sleeve. (1) In the preferred embodiment, the flap (4) is at least the size of the Connection (9) portion of the Sleeve. (1) Alternative embodiment Connections (9) have various configurations of the Connection (9) portions of the flap (4) and Sleeve. (1)

The adhesive portions of the bandage, depending upon the embodiment, may include the flap (4), the inner face (the interior) (7) surface of the Sleeve (1), the Connection (9) portion of the Sleeve (1), are preferably protected and covered with a removable sheet (14) or shield material. In still other embodiments, portions of the inner face (7) surface without adhesive (20) portions may be protected and covered with a removable sheet or shield material. (14) The removable sheet or shield material (14) is preferably a material that is easily detached from the adhesive (20) fastening material. The purpose of the removable sheet or shield material (14) is to protect the adhesive (20) material and/or the medicament (22) until it is peeled away, thereby exposing the adhesive (20) material and/or the medicament (22) in order to function to secure the skin of the person to the desired positioning of the Sleeve (1) and the flap (4) or to help retain and protect the medicament. (22) The material of the removable sheet or shield material (14) is preferably a thin plastic film with sufficient strength to maintain structural integrity when releasable peeled from the surface of the adhesive (20) material or medicament (22), but the sheet or shield material (14) may consist of any suitable material.

The flap (4) may also serve to close wounds, such as breaks or openings in the skin. When skin is broken, even during surgery, germs can enter and cause infection. Some embodiments of the invention can be employed similar to a butterfly closure, wherein these embodiments are designed to pull the edges of a skin wound together and at least partially closed over the wound in order to facilitate healing and closure of the wound. Bringing the wound edges together and keeping them together helps reduce infection rates due to increasing the epidermal barrier. The healing then occurs with replacement growth over the remaining wound opening. Depending upon the embodiment, the flap (4) may bring separated portions of skin together for a wound oriented along the digit line axis (proximal to distal orientation), perpendicular to the digit axis (dorsal to ventral, or palmer to dorsal), in a diagonal orientation, or even in combinations thereof.

Now referring to FIGS. 9 and 10, the fastening method (10) may include an adhesive portion (20), a hook-and-loop portion (11), or other fastening methods. (10) In some embodiments, the flap (4) has a fastening method (12), in other embodiments, the Connection (9) Sleeve (1) portion has the fastening method (10) incorporated in the outer surface (6) of the second end portion (8) of the Sleeve (1) (13), and in yet additional embodiments, both the Sleeve (1) and flap (4) (the Connection (5) portion) have fastening methods (10) incorporated.

Now referring to FIGS. 10, 11, and 12, the inner face surface (7) of the Sleeve (1) may be at least partially coated with a semi-permanent adhesive (20) that allows the Sleeve (1) to stick to the skin but can be removed without damage to the tissue of the body tip. The portions of the Sleeve (1) interior that are not coated with the adhesive (20) may be designed to support an absorbent material (21) that interfaces with a wound. Alternative embodiments of the Sleeve (1) to not contain any adhesive (20) or absorbent material (21) in the inner face portion (7) of the Sleeve. (1) As is the case with most bandages, the absorbent portion (21) is designed to absorb bodily fluids and may also contain medicaments (22) to facilitate in wound healing. The absorbent portion (21) is formed from a material selected from the group consisting of: cloth, fabric, gauze, linen, cotton, other natural materials, synthetic cotton blends, silk, woven paper, water-absorbing hydrogel, or mixtures thereof. Alternative embodiments may use other materials as an absorbent portion. (21) Additionally, a thin, porous polymer coating may cover the absorbent portion to keep it from sticking to the wound in certain embodiments.

In the preferred embodiment, there may small holes (sometimes referred to herein as “small openings”) in one or more of the Sleeve (1), the flap (4), adhesive (20) material, and/or the absorbent material (21) to allow the skin covered by the Sleeve (1), including the wound itself, access to air in order to facilitate healing. In alternative embodiments where a medicament (22) is included, the embodiment may be waterproof or water-resistant, or other reasons may exist where the small openings may be omitted from the material of the Sleeve (1), flap (4), and even the absorbent material (21) side interfacing with the Sleeve (1) at the attachment point (23) of the absorbent material. (21) In still another alternative embodiment, medicament (22) may be used when one or more of the Sleeve (1), the flap (4), adhesive material (20), and/or the absorbent material (21) contains small holes in order to temporarily apply medicament (22) to the wound, but then allow the medicament (22) to escape through the holes over time in order to provide aeration to the affected area once the medicament (22) is substantially gone from the Sleeve (1) bandage.

The invention is a protective bandage shaped as a Sleeve (1) to cover a body tip, with a first end portion (2) and a second end (8) portion, wherein said first end (2) portion is narrower than said second end (8) portion, and said protective bandage Sleeve (1) having an inner face surface (7) and a corresponding outer surface (6), said inner face surface (7) opposite said outer surface. (6) The first end portion (2) being closed and said second end portion (3) being open, said second end (3) having a continuous circumferential edge, which may be disposed below the distal index joint, but disposition of the continuous circumferential edge may vary with different embodiments. The inner face surface (7) designed to receive a body tip and said first end portion (2) designed to receive the end of a body tip. A single flap (4) structure attached to said outer surface (6) at said second end portion (8) and at least partially laminated with an adhesive (20) substance. The protective bandage Sleeve (1) may contain a pad of absorbent material (21) in said inner face surface. (7) The inner face surface (7) may contain a medicament (22) in some embodiments to aid in the healing of the body tip anywhere said protective bandage Sleeve (1) inner surface portion (7) contacts the body tip contained therein. The flap (4) is designed to pull the body tip skin wounds together to close a body tip wound. The flap (4) is designed to secure said protective bandage Sleeve (1) in position by radial attachment to said radial attachment to said outer surface. (6)

The invention has several embodiments in addition to the preferred embodiment, as mentioned in the foregoing. For example, the flap (4) structure is designed to secure said protective Sleeve (1) bandage in position by radial attachment to said outer surface. (6) In another embodiment, the flap (4) incorporates a fastening method (10) designed to attach said flap (4) to said outer surface. (6) An alternative embodiment finds the outer surface (6) incorporating a fastening method (10) designed to attach said flap (4) to said outer surface. (6) Additionally, the outer surface (6) and the flap (4) may both incorporate a fastening method (10) designed to attach said flap (4) to said outer surface. (6)

In another embodiment, the flap (4) is designed to secure said protective bandage Sleeve (1) in position by pulling down the inside-out (32) compression cuff flap (4) to and over said outer surface (6), orienting the compression cuff flap (4) right-side out. (37) Additionally, the protective flap (4) is designed to secure said protective bandage Sleeve (1) in position by pulling down the inverted compression cuff flap (4) to said outer surface (6) and at least partially to said body tip skin beyond said second end portion (8) and the terminal end (36) of said second end portion. (8)

In another embodiment, the protective bandage Sleeve (1) is made of a material with small holes. In still yet another embodiment, the medicament (22) dissipates through said small holes, and air is permitted access to the wound through the small holes. However, in another embodiment, the protective bandage Sleeve (1) and flap (4) are made of a waterproof material.

In another embodiment, the inner face surface (7) is at least partially laminated with an adhesive material. (20) The inner face surface (7) is designed to work with said flap (4) to pull the body tip skin wound edges together to close a body tip wound.

The bandage Sleeve (1) shape to cover a body tip is a shape selected from the group consisting of (a conical frustum with a closed narrow end, a blunted tangent ogive, an elliptical cone, a blunted parabolic cone, a cylinder with a closed end). The bandage sleeve (10) and flap (4) are made of a material selected from the group consisting of (plastic, polyvinyl chloride, polyethylene, polyurethane, latex, woven fabric, rayon, nylon, or a combination thereof). In another embodiment, the inner face surface (7) may contain an absorbent material. (21) The inner face surface (7) may contain an absorbent material (21) selected from the group consisting of (cloth, fabric, gauze, linen, cotton, sild, woven paper, synthetic cotton-blends, or a combination thereof).

Thus, while particular embodiments and applications have been illustrated and described, it is to be understood that the disclosed embodiments are not limited to the precise construction and components disclosed herein. Additionally, variants of additional embodiments are possible. For example, several different embodiments may include each part being a different size and/or shape and/or material. Finally, uses of the present invention are not limited to wounds, as the invention can be used to protect body tips for various reasons. Therefore, the spirit and scope of the appended claims and the concepts taught herein should not be limited to the description of the preferred embodiments and embodiments contained herein.

I claim:

1. A protective bandage, comprising:
a. a bandage shaped to cover a body tip with a first end portion and a second end portion, wherein said first end portion is narrower than said second end portion, and said protective bandage having an inner face surface and a corresponding outer surface, said inner face surface opposite said outer surface;
b. said first end portion being closed and said second end portion being open;
c. said inner face surface designed to receive a body tip and said first end portion designed to receive the end of a body tip;
d. a single flap attached to said outer surface at said second end portion;
e. said flap designed to secure said protective bandage in position by radial attachment to said outer surface;
f. said flap is designed to pull the body tip skin wound edges together to close a body tip wound.

2. The protective bandage of claim 1 wherein said flap incorporates a fastening method designed to attach said flap to said outer surface.

3. The protective bandage of claim 1 wherein said outer surface incorporates a fastening method designed to attach said flap to said outer surface.

4. The protective bandage of claim 1 wherein said outer surface and said flap incorporate a fastening method designed to attach said flap to said outer surface.

5. The protective bandage of claim 1 wherein said flap is designed to secure said protective bandage in position by pulling down said flap in an inside-out compression cuff configuration to and over said outer surface thereby orienting the compression cuff right-side out.

6. The protective bandage of claim 5 wherein said flap is designed to secure said protective bandage in position by pulling down said flap compression cuff at least partially to said body tip skin beyond said outside surface and said second end portion.

7. The protective bandage of claim 1 wherein said protective bandage is made of a material with small holes.

8. The protective bandage of claim 7 wherein said inner face surface has a medicament ointment.

9. The protective bandage of claim 8 wherein said medicament dissipates through said small holes over time.

10. The protective bandage of claim 1 wherein said inner face surface is at least partially laminated with an adhesive material.

11. The protective bandage of claim 1 wherein said inner face surface contains no adhesive material.

12. The protective bandage of claim 1 wherein inner face surface is designed to work with said flap to pull the body tip skin wounds together to close a body tip wound.

13. The protective bandage of claim 1 wherein said protective bandage is made of a waterproof material.

14. The protective bandage of claim 1 wherein said bandage shape to cover a body tip is a shape selected from the group consisting of (conical frustum with a closed narrow end, a blunted tangent ogive, an elliptical cone, a blunted parabolic cone, a cylinder with a closed-end).

15. The protective bandage of claim 1 wherein said bandage is made of a material selected from the group consisting of (plastic, polyvinyl chloride, polyethylene, polyurethane, latex, woven fabric, rayon, nylon, or combination thereof).

16. The protective bandage of claim 1 wherein at least a portion of said inner face surface contains an absorbent material.

17. The protective bandage of claim 16 wherein said inner face surface contains an absorbent material selected from the group consisting of (cloth, fabric, gauze, linen, cotton, silk, woven paper, synthetic cotton blends, or a combination thereof).

18. The protective bandage of claim 1 wherein said second end terminates in a continuous circumferential edge disposed below the distal index joint.

19. A protective digit bandage, comprising:
a. a bandage shaped to cover a digit with a first end portion and a second end portion, wherein said first end portion is narrower than said second end portion, and said protective digit bandage having an inner face surface and a corresponding outer surface, said inner face surface opposite said outer surface;
b. said first end portion being closed and said second end portion being open, said second end having a continuous circumferential edge disposed below the distal index joint;
c. said inner face surface designed to receive a digit and said first end portion designed to receive the end of a body tip;
d. a single flap attached to said outer surface at said second end portion and at least partially laminated with an adhesive substance;
e. said protective digit bandage containing pad in said inner face surface;
f. said flap is designed to secure said protective bandage in position by radial attachment to said outer surface;
g. said flap is designed to pull the body tip skin wounds together to close a body tip wound.

20. The protective bandage of claim 19 wherein said elongate flap is one of the group of consisting of (an elongate flap extending from said second end portion or a flap circumscribing said second end portion in a skirt orientation).

21. The protective bandage of claim 19 wherein said protective bandage is made of a material with small holes.

22. The protective bandage of claim 21 wherein said inner face surface has a medicament ointment.

23. The protective bandage of claim 22 wherein said medicament dissipates through said small holes over time.

24. The protective bandage of claim 19 wherein said inner face surface is at least partially laminated with an adhesive material.

25. The protective bandage of claim 19 wherein said inner face surface contains no adhesive material.

26. The protective bandage of claim 19 wherein said inner face surface is designed to work with said flap to pull the body tip skin wounds together to close a body tip wound.

27. The protective bandage of claim 19 wherein at least a portion of said inner face surface contains an absorbent material.
